(19) United States
(12) Patent Application Publication
(10) Pub. No.: US 2021/0275308 A1
(43) Pub. Date: Sep. 9, 2021

(54) Acetabular Implant for Hip Prosthesis

(71) Applicant: GILES, Caluire-Et-Cuire (FR)

(72) Inventors: Daniel Noyer, Luzinay (FR); Philippe Bauchu, Lyon (FR); Alain Cypres, Saint-Nizier-Sous-Charlieu (FR); Arnaud Fiquet, Caluire-Et-Cuire (FR); Christophe Roy, Chatuzange-Le-Goubet (FR); Olivier Bonnard, Meyzieu (FR); Philippe Girardin, Lezigneux (FR); Bertrand Seutin, La Batie-Rolland (FR); Gualter Vaz, Lyon (FR); Philip John Roberts, Nantwich (GB)

(21) Appl. No.: 16/884,753

(22) Filed: May 27, 2020

(30) Foreign Application Priority Data
Mar. 4, 2020 (FR) 2002164

(51) Int. Cl. A61F 2/34; A61F 2/36

(52) U.S. Cl. CPC A61F 2/34; A61F 2/3609; A61F 2310/00017; A61F 2002/30649; A61F 2310/00239; A61F 2002/3412; A61F 2310/00059

(57) ABSTRACT

An acetabular implant for a hip prosthesis including:
a fixation cup defining an inner cavity having a substantially hemispherical shape, made of polyethylene and having, on its outer wall, means capable of favoring its fixation with cement within the acetabular cavity of the pelvis of the patient to be prosthesized, the inner cavity being coated with an armoring made of a biocompatible material of high hardness, said armoring defining the smooth inner cavity of substantially hemispherical shape intended to receive the mobile insert;
an insert, mobile within said inner cavity and having its smooth outer surface intended to cooperate by ball and socket joint with the inner cavity of the fixation cup, and defining in turn a smooth inner cavity also having a substantially hemispherical shape; said mobile insert being made of high-density polyethylene;
a prosthetic femoral head or prosthetic ball, secured to a femoral stem, of spherical shape and intended to be received in the inner cavity of the mobile insert and to form a second ball and socket joint.

### DOMAIN OF THE INVENTION

The invention relates to an acetabular implant for a hip prosthesis, of dual mobility type.

### BACKGROUND

Complete hip prostheses, called dual mobility prostheses, are now widely used. Chiefly, such a dual mobility principle aims at the rotation of a prosthetic femoral head or prosthetic ball, affixed to a prosthetic stem inserted into the femur, within a mobile insert, the latter being in turn also mobile in rotation within a cup or acetabular implant fixed in the acetabular cavity of the pelvis of the patient to be prosthesized.

Two joints are thus defined, respectively a small joint (prosthetic femoral head/mobile insert) and a large joint (mobile insert/cup), whereby the generic term of dual mobility. Conventionally, the small joint is implemented in motions of small amplitude while the large joint starts functioning when the femoral collar of the prosthetic stem comes into contact with the stepped portion conventionally fitting the mobile insert, in motions of large amplitude.

Dual mobility has enabled to significantly decrease dislocations of such a prosthetic joint. Such a prosthesis has for example been described in document FR 2 710 836 or also FR 2 948 013.

The use of such so-called dual mobility prostheses has significantly decreased the number of dislocations of the joints thus prosthesized and, as a corollary, increased their lifetime. Nevertheless, there remains a number of problems, due to the presence of a double joint surface, respectively of the small joint and of the large joint, and generating, due to the respective contact surfaces and despite the progress made in terms of materials, phenomena of dislocation or also blocking, resulting in a prosthetic failure.

Further, the fixation of the fixation cup within the acetabular cavity systematically is a critical point for the durability of the prosthesis then implanted. Thus, to optimize the fixation of the cup to be affixed, the principle of metallic fixation cups, possibly expansive, provided with fixation means, such as for example fixation tabs, with a large number of variants, has been developed. There is an abundant literature on this subject, but a main tendency concerns the implementation of a systematically metallic fixation cup, which generally results in increased costs of manufacturing, or even of implantation. Such costs thus run up against the current tendency of State agencies or complementary organizations taking charge of such costs.

Further, the fixation cups currently used in the context of dual mobility prostheses have a metallic convexity and are intended to be mainly implanted within the acetabular cavity without cement, and provided, for some of them, in a metallic version to be sealed with cement. However, cups made of polyethylene, a biocompatible material, and intended to be sealed with cement within said acetabular cavity remain to date a reference in the eyes of many orthopedic surgeons throughout the world, which have a sufficient history in terms of modalities of fixation into the bone via cement to appreciate the opportunity of their implementation.

A dual mobility prosthesis, implementing a cup made of polyethylene, attached by cement within the acetabular cavity, and having its mobile insert also made of polyethylene, is also known. However, this mobile insert is armored by means of a metal coating at the level of its external surface, that is, at the level of its area of cooperation by ball-and-socket joint with the inner cavity of the fixation cup. The armoring aims at favoring the functioning of the large joint, since the cooperation of two surfaces made of polyethylene with each other is avoided. Due to such an armoring, the weight of the mobile insert is thus increased, tending to cause its permanent tilting against the stem with which it cooperates via the small joint with the prosthetic ball or prosthetic femoral head affixed to said stem, causing a premature wear of the stepped portion of the mobile insert.

The object of the invention is to provide such a hip prosthesis, more particularly a dual mobility acetabular implant according to the general definition, for a hip prosthesis, on the one hand having a limited cost, and on the other hand enabling to do away with the problems remaining at the level of the two joints forming the dual mobility.

### SUMMARY OF THE SPECIFICATION

Thus, the specification concerns an acetabular implant for a hip prosthesis comprising:

a fixation cup to be cemented in the acetabular cavity of the patient's pelvis, defining an inner cavity of substantially hemispherical shape;

an insert, mobile within said inner cavity and having its smooth outer surface intended to cooperate by ball and socket joint with the inner cavity of the fixation cup, and defining in turn a smooth inner cavity also having a substantially hemispherical shape;

a head or ball, secured to a femoral stem, of spherical shape and intended to be received in the inner cavity of the mobile insert and to form a second ball and socket joint.

More specifically:

the fixation cup is made of polyethylene and has, on its outer wall, outgrowths or protrusions, and generally means capable of favoring its sealing with cement within the acetabular cavity;

the inner cavity of said fixation cup is coated with an armoring made of a material of high hardness, for example, an alloy of chromium-cobalt or oxidized zirconium type, stainless steel, ceramic, PEEK (polyetheretherketone), etc., and generally any biocompatible material of high hardness, said armoring defining the smooth inner cavity having a substantially hemispherical shape intended to receive the mobile insert;

the mobile insert is made of high-density polyethylene.

In other words, the invention comprises first enabling to seal the fixation cup with cement in the acetabular cavity, as already mentioned, made of high-density polyethylene, which sealing operation is eased by the outgrowths or protrusions of its outer surface. It then comprises armoring said fixation cup with a biocompatible material of high hardness, and typically, metal, stainless steel, PEEK, ceramic or an oxidized zirconium alloy of the type commercialized under trade name Oxinium, and this, to optimize the mobility of the mobile insert within the large joint, while in particular limiting the genesis of waste and other defects capable of blocking the joint.

### BRIEF DESCRIPTION OF THE DRAWINGS

The foregoing features and advantages of the present invention will now be discussed in the following non-limiting description of a specific embodiment, in relation with the accompanying drawings.

FIG. 1 is a simplified representation in sagittal cross-section of the acetabular implant according to the invention.

FIG. 2 is an exploded view of the implant of FIG. 1.

FIG. 3 is a simplified perspective view of a specific embodiment of the fixation cup according to the invention.

FIG. 4 is a simplified sagittal cross-section view of the acetabular implant according to another embodiment of the invention.

### DETAILED DESCRIPTION

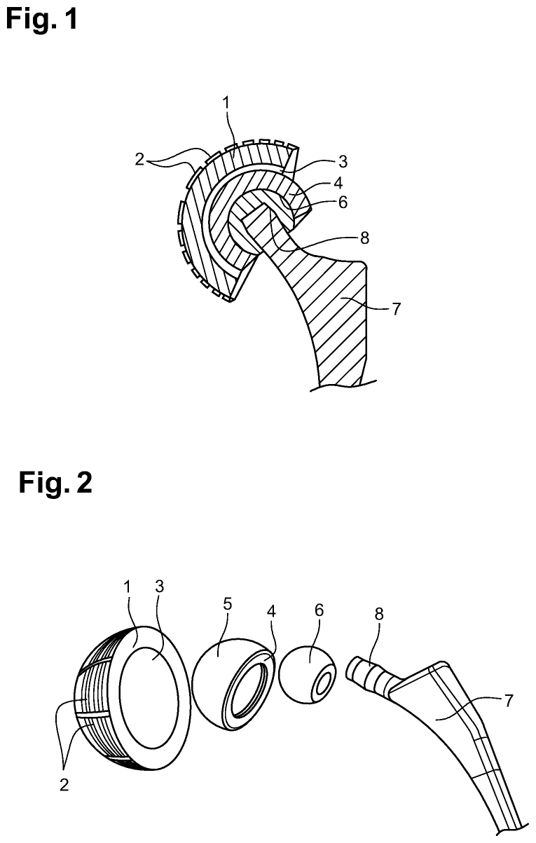

An acetabular implant according to a first embodiment of the invention has thus been shown in relation with FIGS. 1 and 2.

Essentially, it is first formed of a fixation cup (1) made of high-density polyethylene. The fixation cup is provided at it outer surface with fixation means (2), typically formed of protrusions, grooves, and generally of any device capable of favoring the sealing with cement, of said fixation cup within the acetabular cavity of the considered joint.

According to the invention, the fixation cup has a joint armoring (3) at the level of the inner cavity of hemispherical shape that it defines. Typically, the armoring may first be machined, and then embedded within the polyethylene cup. As a variation, it may be embossed, and then overmolded by means of polyethylene forming said cup. The armoring may also be forged, and then machined, before here again being overmolded with the polyethylene.

Typically, the armoring is made of a material of high hardness, such as, for example, steel, particularly stainless steel, or also a chromium-cobalt alloy, or also of ceramic or also of an alloy based on oxidized zirconium, such as commercialized under trade mark Oxinium®. Whatever the nature of the armoring, it is of course of biocompatible nature.

Thereby, the armoring (3) and the polyethylene fixation cup (1) form one and the same part intended to be implanted at the level of the acetabular cavity previously prepared by milling. The fixation is ensured by sealing with surgical cement.

As already mentioned, the armoring (3) takes part in the large joint, that is, between the fixation cup and the mobile insert. The friction torque between the polyethylene mobile insert and the fixation cup, sealed with cement within the acetabular cavity, thus appears between the hard material forming the armoring (3) of the inner cavity of said fixation cup (1) and the polyethylene forming the mobile insert (4). It is known in prior art that the polyethylene/polyethylene friction torque is doomed to fast failure due to poor tribological conditions. The adding of the hard material armoring of the invention first aims at providing the same friction coefficient at the level of the large joint as at that of the small joint (prosthetic ball or prosthetic femoral head placed on the femoral stem, made of metal or ceramic or Oxinium/inner cavity of the mobile insert made of polyethylene).

Further, the forming of an armoring made of a material of high hardness provides a better distribution of the stress in the areas of the acetabular implant made of polyethylene, enabling to do away with creep problems or the like. Said armoring thus acts as a barrier tending to oppose the deformation of the mobile insert, the latter following the shape of the concave portion of said armoring, and the polyethylene cup to be cemented, solid with the armoring, following the shape of the convex portion of the latter, in particular, when the assembly is under load at the time, for example, of the footstrike performing the complete unipodal loading of the implant.

Further, the implantation of such an armoring provides a damping power to the joint thus prosthesized. Indeed, the armoring, of relatively small thickness, typically in the range from approximately 0.8 to 3 millimeters, without for this accuracy to limit the invention, but depending on the characteristics of the material used, is present once the implant has been installed, between two polyethylene thicknesses. Thus, for example, at the strike of the concerned foot, the generated pressure first tends to deform the mobile insert which thereby fully bears against the armoring. The latter then restores this load to the portion of the polyethylene fixation cup. As a result, in addition to the effects inherent to the implementation of the dual mobility principle, which preserves from the shearing effects of the acetabular fixation, due to the decoupling of the frictions implemented during motions, the cemented fixation is preserved due to this damping, thus contributing to a notable benefit for the patient.

The inner cavity defined by the concave joint armoring (3) is intended to receive a mobile insert (4) made of high-density polyethylene, having a convex and hemispherical outer shape (5). The outer diameter of the mobile insert (4) corresponds, to within the play, to the inner diameter of the concave armoring (3). A first joint surface between the fixation cup (1) and the mobile insert (4) is thus defined.

The latter (4) also defines an inner cavity, here again of hemispherical shape, intended to cooperate by ball and socket joint with a femoral head or ball (6) made of steel, or even of ceramic or of oxidized zirconium, here again of biocompatible quality. The outer diameter of the prosthetic ball (6) here again corresponds, to within the play, to the inner diameter of the mobile insert (4).

The prosthetic femoral head or prosthetic ball is intended, in known fashion, to be affixed at the end of a femoral step (7), for example, provided with a Morse taper (8) and this, in known fashion. The use of a monoblock femoral tern with a head of adapted size may however be envisaged.

Due to the design of such a particular implant, it can be understood that the operation of the joint is optimized and, as a corollary, a greater comfort is provided to the patient.

Further, due to the dual mobility and to the two polyethylene layers, the cemented fixation is less stressed, particularly in shearing and compression, providing a better life expectancy to the bone fixation, and thus, as a corollary, to the prosthesis thus implanted.

Further, the polyethylene forming the fixation cup or the mobile insert is no longer submitted to a friction with an element also made of polyethylene due to the armoring, and thus, as a corollary, is no longer submitted to the subsequent wear, and there is no further release of wearing particles within the joint and within the periarticular bone.

Moreover, such an implant is more easily implantable at the level of an acetabular cavity capable of exhibiting a significant bone defect, since said bone defect may be filled with the cup fixation cement. In the same line of thought, such an implant may also be cemented within an acetabular reconstruction ring (such as a Kerboull cross, a Ganz or Muller or Bursch-Schneider ring, or any other metallic acetabular reconstruction system), particularly in the case of a situation of precariousness of the acetabular bone stock.

Finally, the cost of the forming of the implant of the invention is decreased due to the nature of the materials used and of their implementation modes.

It should further be specified that by combining the teachings of the present invention with those described in French patent application FR 1902133, unpublished at the date of the filing of the present application, the reliability of the considered implant is optimized, on the one hand, due to the results inherent to the present invention and, on the other hand, by the decreasing of the torque difference between the small joint and the large joint consecutive to the features described in this other application.

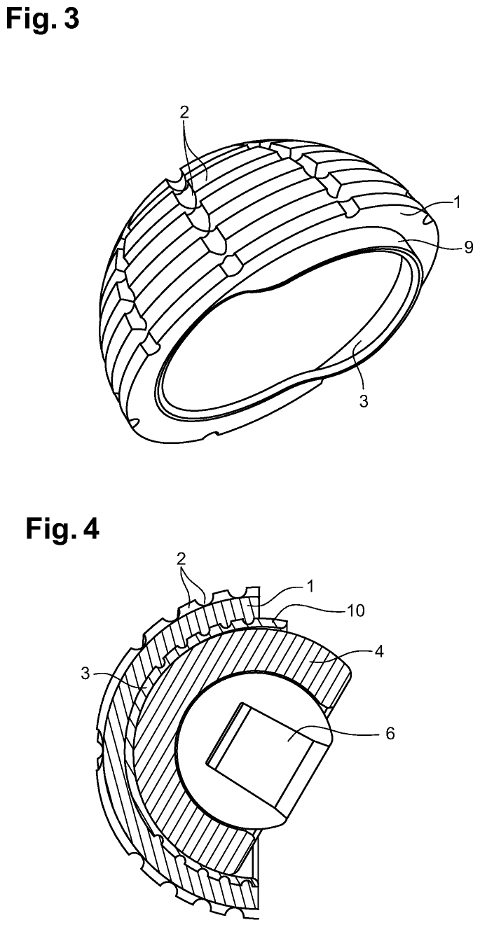

In the embodiment illustrated in FIGS. 1 and 2, the acetabular fixation cup is free of any anti-dislocation rim, as usual on standard so-called dual mobility prostheses. Another embodiment of the present invention has been shown in FIG. 3, including such an anti-dislocation rim (9). This rim (9) may have different possible designs, and especially result either from the armoring (3), or from the actual cup (1), or from the two assemblies, or even be added. Thereby, in the first three cases, it is monoblock and is made of the material forming the element (cup or armoring) from which it comes, that is, either of polyethylene, or of a biocompatible material of high hardness of the type of those previously mentioned.

Further, in certain rarer pathologies, such as those exhibiting significant muscular deficiencies, it may be appropriate for the surgeon to be able to make the large joint retentive, that is, to prevent the decoaptation of the mobile insert (4) outside of the hemispherical internal cavity defined by the armoring (3). This specific configuration enables to have a substantially revolute joint with possibly some potential translation to authorize articular pumping, that is, due to the play existing within each of the small and large joints, the normal functioning of the joint thus prosthesized. This enables the patient to have a prosthetic joint with a large angular clearance, inherent to the dual mobility with all the other above-listed advantages, while limiting the risk of dislocation capable of resulting from his/her muscular deficiency.

A so-called retentive embodiment of the dual mobility acetabular implant of the invention has thus been shown in FIG. 4. Therein, the fixation cup (1) comprises means for retaining the mobile insert (4), capable of freely allowing circumduction motions, but of opposing translation motions of said mobile insert. Typically, such retention means result from the armoring (3), which extends beyond the equator of the half-sphere that it defines, thus forming a peripheral rim (10), capable of opposing the withdrawal of the mobile insert (4). The latter, previously fitted with its prosthetic femoral head, is force-fit into the fixation cup provided with its armoring.

1. An acetabular implant for a hip prosthesis comprising:
a fixation cup in an acetabular cavity of a pelvis of a patient to be prosthesized, defining an inner cavity of substantially hemispherical shape and having an outer wall;
an insert, mobile within said inner cavity and having a smooth outer surface intended to cooperate by ball and socket joint with the inner cavity of the fixation cup, and defining in turn a smooth inner cavity also having a substantially hemispherical shape;
a prosthetic femoral head or prosthetic ball, secured to a femoral stem, said head or ball being of spherical shape and being intended to be received in the inner cavity of the mobile insert and to form a second ball and socket joint,
wherein the fixation cup is made of polyethylene and has, on its outer wall, outgrowths or protrusions, and generally means capable of favoring a fixation of the fixation cup with cement within the acetabular cavity of the pelvis of the patient to be prosthesized;
wherein the inner cavity of said fixation cup is coated with an armoring made of a biocompatible material of high hardness, said armoring defining the smooth inner cavity of substantially hemispherical shape intended to receive the mobile insert;
and wherein the mobile insert is made of high-density polyethylene.

2. The acetabular implant for a hip prosthesis according to claim 1, wherein the material forming the armoring is made of metal, of stainless steel, of chromium-cobalt alloy, of ceramic, of PEEK, or of an oxidized zirconium alloy.

3. The acetabular implant for a hip prosthesis according to claim 1, wherein the fixation cup is provided with additional anti-dislocation means, monoblock with said fixation cup.

4. The acetabular implant for a hip prosthesis according to claim 1, wherein the fixation cup is provided with additional anti-dislocation means, added on said fixation cup.

5. The acetabular implant for a hip prosthesis according to claim 3, wherein the additional anti-dislocation means (9) are made of polyethylene or of a biocompatible material of high hardness.

6. The acetabular implant for a hip prosthesis according to claim 4, wherein the additional anti-dislocation means (9) are made of polyethylene or of a biocompatible material of high hardness.

7. The acetabular implant for a hip prosthesis according to claim 1, wherein the fixation cup comprises means for retaining the mobile insert, said means being capable of allowing free circumduction motions, but of opposing translation motions of said mobile insert.

8. The acetabular implant for a hip prosthesis according to claim 7, wherein the means for retaining the mobile insert are formed by an extension of the armoring beyond the half-sphere defined by said mobile insert.
